(19) United States
(12) Patent Application Publication
(10) Pub. No.: US 2019/0021898 A1
(43) Pub. Date: Jan. 24, 2019

(54) MENSTRUAL CUP

(71) Applicant: EASE AND MORE, Seoul (KR)

(72) Inventor: Jihye AHN, Gwangju-si (KR)

(73) Assignee: EASE AND MORE, Seoul (KR)

(21) Appl. No.: 16/029,797

(22) Filed: Jul. 9, 2018

(30) Foreign Application Priority Data
Jul. 21, 2017 (KR) 20-2017-0003826

(51) Int. Cl. A61F 5/441; A61F 5/455

(52) U.S. Cl. CPC A61F 5/441; A61F 5/4553

(57) ABSTRACT

The object of the present invention is providing a menstrual cup which may reduce the leakage of menstrual blood by forming a first protrusion outward at an edge of an upper end of a body and bringing the body with improved restoring force into close contact with the inner wall of the vagina. The menstrual cup of the present invention may comprise a body, which is opened at one end to form a storage space inside; a handle formed at the other end of the body, wherein outer diameter of the body may gradually increase from one point between the one end and the other end toward the one end and the other end.

### CROSS-REFERENCE TO RELATED APPLICATION(S)

This application claims priority to Korean Patent Application No. 20-2017-0003826 filed on Jul. 21, 2017, the entire contents of which are herein incorporated by reference.

### TECHNICAL FIELD

The present invention relates to a menstrual cup, and more particularly, to a menstrual cup having a storage space for accommodating menstrual blood therein and inserted and used in a woman's vagina.

### BACKGROUND

Generally, female sanitary products are mainly composed of a pad type sanitary napkin which is attached to the inside of an underwear (panty), and an insertion type sanitary napkin which is inserted into a woman's vagina.

Pad-type sanitary napkin generally has advantages such as excellent absorbency and thin thickness, however, since it is not fixed firmly to underwear at the time of activity but moves easily, there are restrictions on free activities and exercise, and especially, it is necessary to consciously control movement even during sleep.

In addition, the pad type sanitary napkin is known to have a problem of causing skin diseases or itching, because the surface of the sanitary napkin that is brought into contact with the skin when it is worn is made of a chemical material.

Since the insertion type sanitary napkin effectively prevents the leakage of the menstrual blood compared with the pad type sanitary napkin, there is an advantage that the free activities are possible even wearing the insertion type sanitary napkin during menstruation period. However, it is known that harmful substances generated in the insertion type sanitary napkin made of a chemical component can cause various diseases.

Prior arts: Korean Patent No. 10-1726672 and Korean Patent Application Publication No. 10-2006-0062677.

### SUMMARY

The object of the present invention is providing a menstrual cup which may reduce the leakage of menstrual blood by improving restoring force of the body and bringing the body into close contact with the inner wall of the vagina.

The menstrual cup according to one embodiment of the present invention may comprise a body 110, which is opened at one end to form a storage space 111 inside; a handle 120 formed at the other end of the body 110, wherein outer diameter of the body 110 gradually increases from one point between the one end and the other end toward the one end and the other end.

In one embodiment, thickness of the body 110 may become gradually thicker from one point between the one end and the other end toward the one end and the other end.

In one embodiment, a first protrusion 112 may be formed along an edge of one end of the body 110 and second protrusions 121 may be formed by a predetermined interval in a longitudinal direction on an outer circumferential surface, wherein a maximum outer diameter of the first protrusion 112 may be larger than the maximum outer diameter of the other end of the body 110.

The menstrual cup according to another embodiment of the present invention, may comprise a body 110, which is opened at one end to form a storage space 111 inside; a handle 120 formed at the other end of the body 110, wherein an expansion space 115 may be formed in the first protrusion 112, an injection pump 130 for injecting air into the expansion space 115 and a discharge valve 132 for discharging the air injected into the expansion space 115 to the outside are formed at the handle 120.

Outer diameter of the body of the menstrual cup according to another embodiment of the present invention, outer diameter of the body 110 may become gradually wide from one point between the one end and the other end toward the one end and the other end.

The menstrual cup of the present invention as described above may have the following effects.

An advantageous effect that when the body is folded and inserted into the vagina the size of the insertion direction portion is reduced to facilitate insertion, while the size of the hand-held portion is wide so as to minimize the slip may be achieved.

An advantageous effect that the restoring force of the body is improved, and the body is brought into close contact with the inner wall of the vagina, thereby reducing leakage of menstrual blood may be achieved.

When the body 110 is inserted into the vagina, one end and the other end of the body 110 are in contact with the inner wall of the vagina, while the middle portion of the body 110 is spaced apart from the inner wall of the vagina, thereby minimizing the friction between the body 110 and the vaginal wall. As a result, an advantageous effect of minimizing damage to the vaginal wall and various side effects may be achieved.

### BRIEF DESCRIPTION OF THE DRAWINGS

FIG. 1 is a perspective view of a menstrual cup according to an embodiment of the present invention at one view point.

FIG. 2 is a perspective view of a menstrual cup according to an embodiment of the present invention at another view point.

FIG. 3 is a cross-sectional view of a menstrual cup according to an embodiment of the present invention.

FIG. 4 is a cross-sectional view of a sanitary cup according to another embodiment of the present invention.

FIG. 5 is a perspective view to describe an operation of the discharge valve of the menstrual cup according to another embodiment of the present invention.

### DETAILED DESCRIPTION

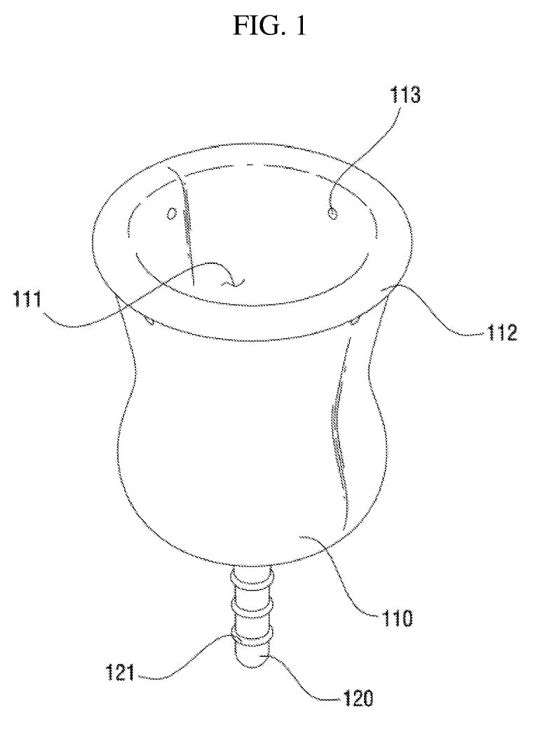

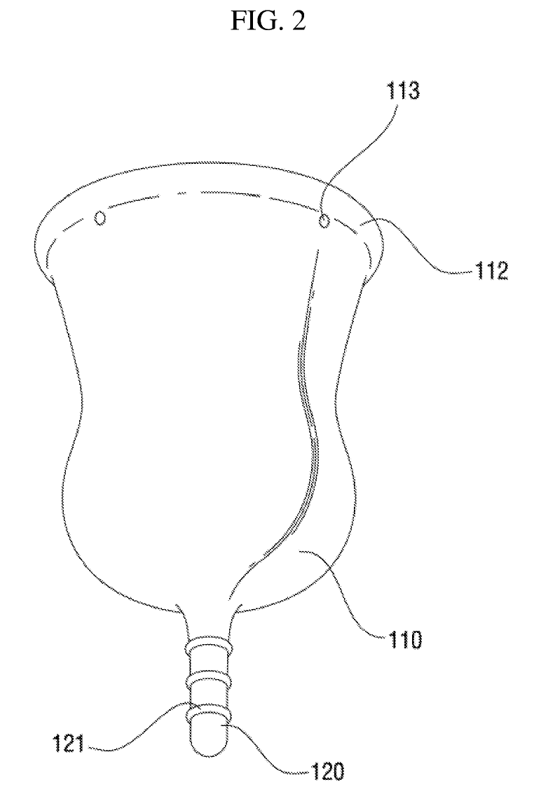

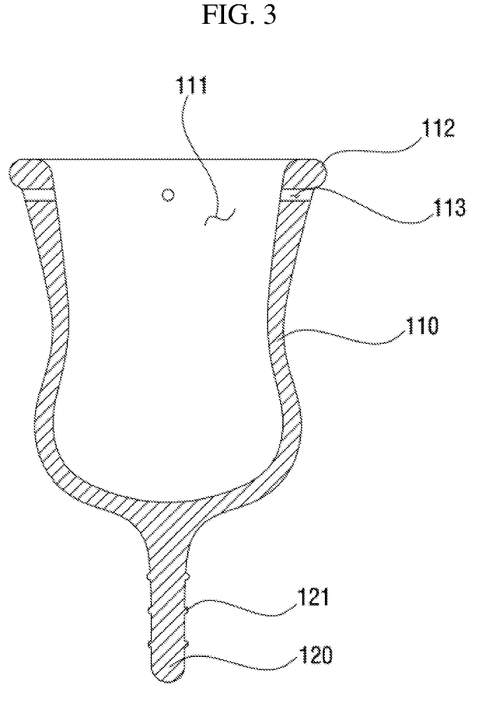

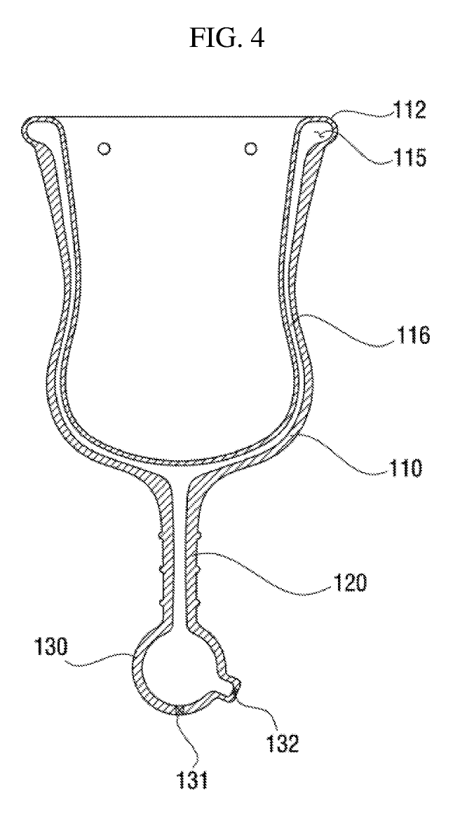

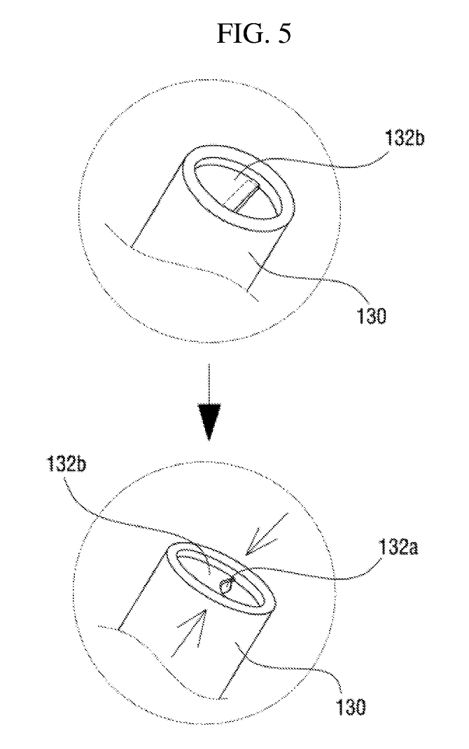

FIG. 1 is a perspective view of a menstrual cup according to an embodiment of the present invention at one view point. FIG. 2 is a perspective view of a menstrual cup according to an embodiment of the present invention at another view point. FIG. 3 is a cross-sectional view of a menstrual cup according to an embodiment of the present invention. FIG. 4 is a cross-sectional view of a sanitary cup according to another embodiment of the present invention. FIG. 5 is a perspective view to describe an operation of the discharge valve of the menstrual cup according to another embodiment of the present invention.

As depicted in FIGS. 1 to 3, the menstrual cup according to the embodiment of the present invention may comprise a body 110 and a handle 120.

The body 110 may be made entirely of a silicon material, and one end of the body 110 may be opened like a cup to form a storage space 111 that can store menstrual blood therein.

The body 110 may be formed such that outer diameter of the body 110 gradually increases from one point between the one end and the other end toward the one end and the other end.

The one point between the one end and the other end of the body 110 may be a center of the body 110 in the longitudinal direction. The body 110 may be formed such that its outer diameter gradually decreases from one end to the center and gradually increases from the center to the other end.

In other words, the body 110 may have a shape in which the center portion is recessed inward like a mortar.

Here, the outer circumferential surface and the inner circumferential surface of the body 110 may be formed as inclined surfaces from one end to the center and formed as curved surfaces of arc shape from the center to the other end.

That is, as depicted in FIG. 3, as viewed cross section, a side surface from the one end, which is the upper end, to the center may be formed as an inclined surface with gradually decreasing outer diameter, and a side surface from the center to the other end, which is the lower end, may be formed as a curved surface with gradually increasing outer diameter.

As described above, since the body 110 may have a shape in which its outer diameter gradually decreases as it goes toward the center, when the body 110 is inserted into the vagina, the one end and the other end of the body 110 may contact with the inner wall of the vagina and the center portion of the body 110 may be slightly spaced apart from the inner wall of the vagina, thereby minimizing the friction between the body 110 and the vaginal wall. As a result, an advantageous effect of minimizing damage to the vaginal wall and various side effects may be achieved.

Furthermore, the body 110 may be formed such that thickness of the body 110 becomes gradually thicker from one point between the one end and the other end toward the one end and the other end.

That is, the thickness of the body 110 may gradually decrease from the one end to the center and may gradually increase from the center to the other end again.

By making the thickness of the one end of the body 110 thicker than the middle of the body 110, the restoring force of the body 110 may be improved for the body 110 to be expanded well inside of the vagina into as an original shape. Further, by making the thickness of the center portion of the body 110 thinner than the one end and the other end of the body 110, when the body 110 is folded and inserted into the vagina, the body 110 may be folded well. Further, by making the thickness of the other end thick again, when the other end portion of the body 110 is held by hand and the body 110 is inserted into the vagina, modifying of a hand-held portion of the body 110 may be minimized.

According to the body 110 above, when the body 110 is folded and inserted into the vagina, since the size of the one end, which is the insertion direction portion, is reduced, an advantageous effect that it is easy to insert the body 110 into the vagina may be achieved, while, since the size of the other end, which is held by hand, is wide, an advantageous effect of minimizing the slipping may be achieved.

As another mean for preventing the menstrual cup from slipping in the hand when inserting into the vagina, the lower end of the body 110 (i.e., the lower portion of one point between one end and the other end having the smallest outer diameter) may be coated with a material different from a material of the body 110. That is, the menstrual cup according to one embodiment of the present invention may comprise a coating layer positioned at the lower end of the body 110 and contacting the body 110 and made of the material different from the material of the body 110.

Meanwhile, a first protrusion 112 may be formed on the outer circumferential surface of the one end along the circumference.

The first protrusion 112 may be a projection protruding in a ring shape along an edge of the one end of the body 110, consist of one, and be formed such that a maximum outer diameter of the first protrusion 112 is larger than a maximum diameter of the other end of the body 110.

That is, the body 110 may be formed such that the outer diameter of the upper end is the largest.

The first protrusion 112 above, when the body 110 is inserted into the vagina, may most tightly contact with the inner wall of the vagina to prevent menstrual blood from leaking to the outside and to prevent the body 110 from slipping.

As describe above, since the first protrusion 112 may be formed on the outer circumferential surface of the body 110, an advantageous effect of minimizing the leakage of menstrual blood and improving the fixation force between the body 110 and the inner wall of the vagina may be achieved.

Furthermore, by the first protrusion 112 above, the thickness and the strength of the one end of the body 110 may become thicker and stronger than the other end of the body 110.

Generally, the body 110 may be folded into a ¼ size to for insertion into the vagina and then expanded to its original shape by the restoring force after the insertion into the vagina.

According to the prior arts, although, the thickness of the body 110 is formed to be thin to fold the body 110 easily, the restoring force is reduced as the thickness of the body 110 being thin. As a result, the tightness between the body 110 and the inner wall of vagina is reduced, thereby, causing the leakage of menstrual blood.

On the other hand, according to an embodiment of the present invention, since the menstrual cup may comprise the first protrusion 112 formed on the one end of the body 110, the thickness of the one end of the body 110 may be thickened to increase the strength and the thickness of the remaining portion of the body 110 may be reduced for easy folding and improved restoring force, also.

Meanwhile, opening holes 113 penetrating from inside to outside may be formed on one side portion of the one end of the body 110.

The opening holes may be four, and may be spaced apart from each other along the outer circumferential surface of the body 110.

Furthermore, the opening holes 113 may be disposed adjacent to the lower side of the first protrusion 112.

The opening holes 113 may be passages thorough which air flows into the storage space 111, when the body 110 is restored after the body 110 is folded and inserted into the vagina

As described above, since the opening holes 113 may be formed in the body 110, when the body 110 is restored, an advantageous effect of facilitating the inflow of the air into the storage space 111 and improving the restoring force may be achieved.

Meanwhile, the handle 120 may be formed to be elongated in the longitudinal direction at the other end of the body 110, so as to be easily held and taken out by hand for pulling out the body from the inside of the vagina.

The handle 120, like the body 110, may be made of silicon, which is a soft ductile material, and may have a diameter of 5 to 7 mm, a length of 2 to 4 cm, and a rounded end.

Second protrusions 121 may be formed on the outer circumferential surface of the handle 120 by a predetermined interval in the longitudinal direction.

The second protrusions 121 may be projections protruding in a ring shape along the circumference of the handle 120, and may be three.

As described above, since the second protrusions 121 may be formed on the handle 120, an advantageous effect of preventing the handle 120 from slipping, when the handle 120 is held and pulled, and improving grip feeling may be achieved.

Meanwhile, in some cases, an expansion space 115, into which the air is injected, may be formed in the one end of the body 110.

Specifically, as depicted in FIG. 4, the expansion space 115, which is an empty space, may be formed in a band shape along the circumference in the one end of the body 110, at which the first protrusion 112 is formed, an injection pump 130 for injecting air into the expansion space 115 may be formed at an end of the handle 120.

Obviously, a passage 116 through which the air moves may be formed between the expansion space 115 and the injection pump 130.

The injection pump 130 may be a circular sphere shape, in which an empty space connected to the passage 116 is formed. When the injection pump 130 is pressed, the inside air may move into the expansion space 115 and expand the one end of the body 110.

Furthermore, a check valve 131 and a discharge valve 132 may be formed in the injection pump 130, respectively.

The check valve 131 may be formed at an end of the injection pump 130. When the injection pump 130 is pressed, the check valve 131 is cut off, and, when the force applied to the injection pump 130 is removed, the check valve 131 may be opened, thereby the outside air is sucked into the injection pump 130.

The check valve 131 may be formed in a flat plate shape and may be configured to be openable and closable in a direction in which one end is hinged to introduce air.

Since the check valve 131 is widely used, detailed description thereof will be omitted.

Furthermore, the discharge valve 132 may be always kept closed regardless of the state in which the injection pump 130 is pressed and may be opened when the discharge valve 132 is pressed in a squeeze manner around the discharge valve 132.

Specifically, as depicted in FIG. 6, the discharge valve 132 may be formed to be protruding from a side portion of the injection pump 130 and to have a circular through-hole 132a inside, and two wings 132b may be formed on the inner side of the through-hole 132a and the end portions of the wings 132b may be arranged in contact with each other.

When both sides of the discharge valve 132 are pressed in a squeeze manner, both ends of the wings 132b may be widened, and the discharge valve 132 may be opened.

As described above, by injecting air into the expansion space 115 by the injection pump 130, the one end of the body 110 may be expanded, so that the body 110 may be brought into close contact with the inner wall of the vagina. As a result, an advantageous effect of tightness between the body 110 and the inner wall of vagina may be improved.

The present invention is not limited to the embodiments described above, and can be modified in various ways by those skilled in the art without departing from the spirit and a range of the scope of the following claims. Thus, such modifications are to be construed as being within the scope of this invention.

What is claimed is:

1. A menstrual cup, comprising:
a body of a silicon material, which is opened at one end to form a storage space inside, configured to form a first protrusion protruding outward along an edge of the opened one end side and form a plurality of opening holes, which are spaced apart by a predetermined interval, on a lower side outer circumferential surface of the first protrusion; and
a handle, which is formed integrally and protrudes by a predetermined length from the other end of the body, configured to form second protrusions by a predetermined interval in a longitudinal direction on an outer circumferential surface,
wherein the body is a mortar shape in which outer diameter of the body gradually increases from a center portion between the one end and the other end toward the one end and the other end, and
wherein a maximum outer diameter of the first protrusion is larger than a maximum outer diameter of the other end side of the body.

2. The menstrual cup according to claim 1, further comprising:
an expansion space formed in the first protrusion on the opened one end side of the body;
an injection pump formed at an end of the handle and configured to inject air into the expansion space through a passage formed along the body; and
a discharge valve formed at one end of the injection pump and configured to discharge the air injected into the expansion space to the outside.
